(19) United States
(12) Patent Application Publication
(10) Pub. No.: US 2020/0268447 A1
(43) Pub. Date: Aug. 27, 2020

(54) MEDICAL LASER IRRADIATION DEVICE AND MEDICAL LASER IRRADIATION METHOD

(71) Applicant: SONY CORPORATION, TOKYO (JP)

(72) Inventors: KOICHIRO KISHIMA, KANAGAWA (JP); TAKUYA KISHIMOTO, TOKYO (JP)

(21) Appl. No.: 16/468,409

(22) PCT Filed: Oct. 16, 2017
(86) PCT No.: PCT/JP2017/037401

§ 371 Date: Jun. 11, 2019

(30) Foreign Application Priority Data
Dec. 27, 2016 (JP) 2016-252589

(51) Int. Cl. A61B 18/22; H01S 5/40; H01S 5/022; H01S 5/042

(52) U.S. Cl. CPC A61B 18/22; H01S 5/4087; A61B 2018/00345; H01S 5/042; H01S 5/4012; H01S 5/02284

(57) ABSTRACT

[Object] There is provided .a medical laser irradiation device and a medical laser irradiation method, both of which are capable removing plaque adhering to the angioendothelium more safely using laser light. [Solution] A medical laser irradiation device according to the present disclosure includes a first laser light source configured to emit a first laser light having a wavelength band that is selectively absorbed by plaque existing inside a blood vessel of a living body; a second laser light source configured to emit a second laser light having a wavelength band that is selectively absorbed by the plaque calcified and existing inside the blood vessel; and an optical fiber configured to coaxially guide the first laser light and the second laser light, and at least a part of which is inserted into the blood vessel.

### TECHNICAL FIELD

The present disclosure relates to a medical laser irradiation device and a medical laser irradiation method.

### BACKGROUND ART

Ischemic heart diseases such as angina and myocardial infarction are diseases in which an accumulation of lipids called plaque adhere to the interior of a coronary artery causing a constriction or blocking of the coronary artery, inducing symptoms such as chest pain due to an insufficient supply of blood to the cardiac muscles. Particularly, the onset of an acute myocardial infarction may be life-threatening to the patient, and performing appropriate treatment rapidly is important. One method of treating such ischemic heart diseases is a cardiac catheter treatment called percutaneous coronary intervention (PCI).

One method of removing plaque from the interior of a coronary artery is a treatment method that introduces a high-speed drill called a rotablator up to the lesion through a catheter, and resect the stenotic lesion of the coronary artery. However, with this method, in the case in which the rotablator contacts the angioendothelium, there is a risk of damaging the angioendothelium. Also, with this method, application is difficult for coronary lesions that are completely blocked.

Accordingly, a treatment method that uses an excimer laser emitting ultraviolet light at a wavelength of 308 nm to remove plaque adhering to the interior of a coronary artery (excimer laser coronary angioplasty (ECLA)) has come to be performed (for example, see Non-Patent Literature 1 below). Unlike infrared light at a wavelength of approximately 1 μm, the ultraviolet light at a wavelength of 308 nm radiated from the excimer laser does not produce heat, thereby making it possible to remove plaque more safely.

### CITATION LIST

### Non-Patent Literature

Non-Patent Literature 1: J. Rawlins, S. Talwar, M. Green and P. O'Kane, “Optical coherence tomography following percutaneous coronary intervention with Excimer laser coronary atherectomy”, Gardiovascular Revascularization Medicine, 15(2014), p 29-34.

### DISCLOSURE OF INVENTION

### Technical Problem

However, ultraviolet light at a wavelength of 308 nm is light that is also absorbed by the angioendothelium and the like that one does not want to influence originally. On the other hand, in PCI, because the physician does not perform the treatment operations while actually confirming the affected area with the naked eye, and instead performs the treatment operations while looking at a fluoroscopic image taken with X-rays, there is an undeniable possibility that excimer laser may irradiate an unintended area. For this reason, in the case in which the excimer laser irradiates the angioendothelium or the like, there is a possibility of damaging the angioendothelium.

For this reason, technology capable of removing plaque adhering to the angioendothelium more safely using laser light is strongly desired at present.

Accordingly, in light of the above circumstances, the present disclosure proposes a medical laser irradiation device and a medical laser irradiation method capable of removing plaque adhering to the angioendothelium more safely using laser light.

### Solution to Problem

According to the present disclosure, there is provided a medical laser irradiation device including: a first laser light source configured to emit a first laser light having a wavelength band that is selectively absorbed by plaque existing inside a blood vessel of a living body; a second laser light source configured to emit a second laser light having a wavelength band that is selectively absorbed by the plaque calcified and existing inside the blood vessel; and an optical fiber configured to coaxially guide the first laser light and the second laser light, and at least a part of which is inserted into the blood vessel.

In addition, according to the present disclosure, there is provided a medical laser irradiation method including: guiding a first laser light emitted from a first laser light source configured to emit the first laser light having a wavelength band that is selectively absorbed by plaque existing inside a blood vessel of a living body and a second laser light emitted from a second laser light source configured to emit the second laser light having a wavelength band that is selectively absorbed by the plaque calcified and existing inside the blood vessel with an optical fiber configured to coaxially guide the first laser light and the second laser light; and irradiating the inside of the blood vessel with at least any one of the first laser light or the second laser light from a front end of the optical fiber, at least a part of which is inserted into the blood vessel.

According to the present disclosure, at least one of first laser light having a wavelength band that is selectively absorbed by plaque or second laser light having a wavelength band that is selectively absorbed by calcified plaque is radiated from the front end of an optical fiber of which at least a part is inserted into a blood vessel.

### Advantageous Effects of Invention

According to the present disclosure as described above, it becomes possible to remove plaque adhering to the angioendothelium more safely using laser light.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram schematically illustrating one example of the configuration of a medical laser irradiation device according to an embodiment of the present disclosure.

FIG. 2 is an explanatory diagram for explaining an irradiation unit provided in the medical laser irradiation device according to the embodiment.

FIG. 3A is an explanatory diagram schematically illustrating one example of the configuration of the irradiation unit according to the embodiment.

FIG. 3B is an explanatory diagram schematically illustrating one example of the configuration of the irradiation unit according to the embodiment.

FIG. 4A is an explanatory diagram for explaining a guide wire insertion hole provided in the medical laser irradiation device according to the embodiment.

FIG. 4B is an explanatory diagram for explaining a guide wire insertion hole provided in the medical laser irradiation device according to the embodiment.

FIG. 5 is a block diagram schematically illustrating one example of the configuration of a computational processing unit provided in the medical laser irradiation device according to the embodiment.

FIG. 6 is a block diagram schematically illustrating one example of the optical configuration of the medical laser irradiation device according to the embodiment.

FIG. 7 is a block diagram schematically illustrating one example of the hardware configuration of the computational processing unit according to the embodiment.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that the description will proceed in the following order.

1. Embodiment
1.1. Configuration of Medical Laser Irradiation Device
   1.1.1. Overall Configuration of Medical Laser Irradiation Device
   1.1.2. Irradiation Unit
   1.1.3. Guide Wire Insertion Hole
   1.1.4. Configuration of Computational Processing Unit
   1.1.5. Optical Block Diagram
1.2. Hardware Configuration of Computational Processing Unit

2. Conclusion

### Embodiment

<Configuration of Medical Laser Irradiation Device>

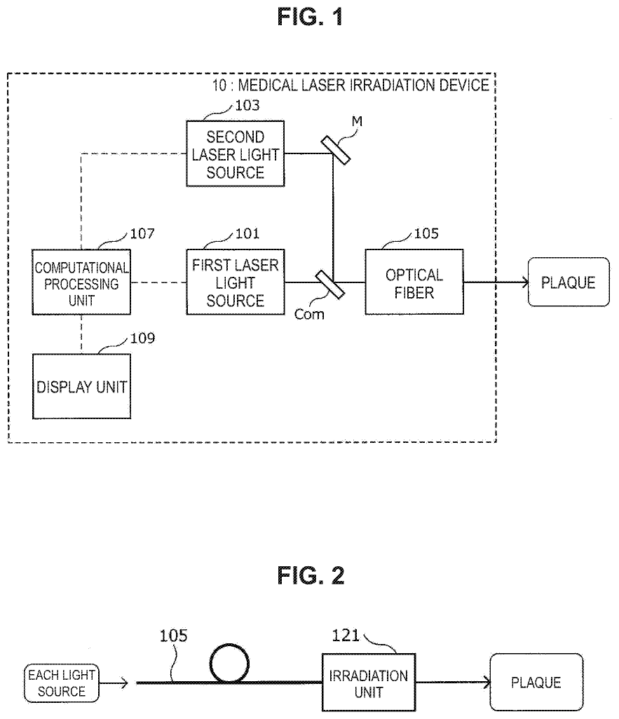

Hereinafter, first, a configuration of an medical laser irradiation device according to an embodiment of the present disclosure will be described in detail with reference to FIGS. 1 to 5.

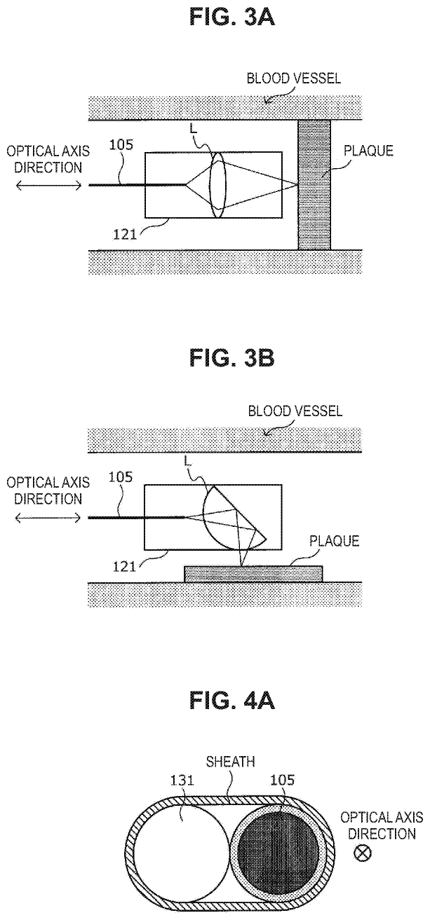

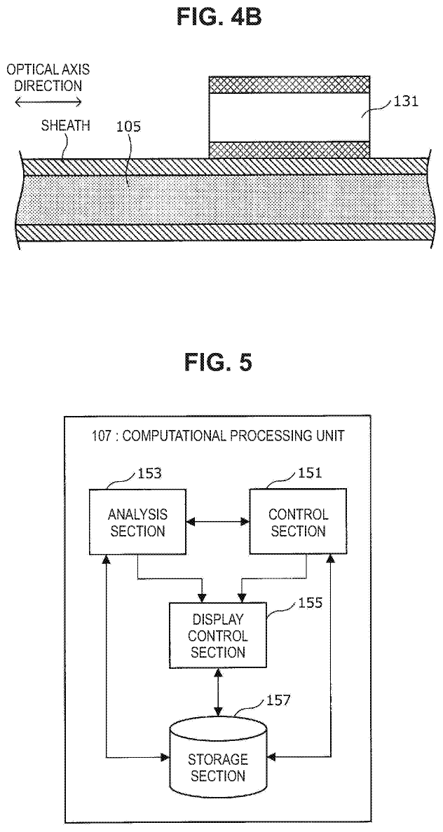

FIG. 1 is an explanatory diagram schematically illustrating one example of the configuration of a medical laser irradiation device according to the present embodiment. FIG. 2 is an explanatory diagram for explaining an irradiation unit provided in the medical laser irradiation device according to the present embodiment. FIGS. 3A and 3B are explanatory diagrams schematically illustrating one example of the configuration of an irradiation unit according to the present embodiment. FIGS. 4A and 4b are explanatory diagrams for explaining a guide wire insertion hole provided in the medical laser irradiation device according to the present embodiment. FIG. 5 is a block diagram schematically illustrating one example of the configuration of a computational processing unit provided in the medical laser irradiation device according to the present embodiment.

### [Overall Configuration of Medical Laser Irradiation Device]

The medical laser irradiation device 10 according to the present embodiment is a device that removes plaque from the interior of a blood vessel by irradiating plaque, that is, an accumulation of lipids existing inside a blood vessel of a living body, with laser light of a predetermined wavelength. As illustrated schematically in FIG. 1 for example, such a medical laser irradiation device 10 is provided with at least a first laser light source 101, a second laser light source 103, and an optical fiber 105. Also, in addition to the above configuration, it is preferable for the medical laser irradiation device 10 according to the present embodiment to additionally include a computational processing unit 107 and a display unit 109.

It has been clearly established that plaque existing inside a blood vessel and plaque with advanced calcification inside a blood vessel selectively absorbs light of predetermined wavelengths. Accordingly, in the medical laser irradiation device 10 according to the present embodiment, by appropriately using light (laser light) with two varieties of wavelengths, plaque and plaque with advanced calcification existing inside a blood vessel are removed effectively.

The first laser light source 101 is a light source that emits the light of a first laser having a wavelength band that is selectively absorbed by plaque existing inside a blood vessel (hereinafter also simply called the “first laser light”). Also, the second laser light source 103 is a light source that emits the light of a second laser having a wavelength band that is selectively absorbed by calcified plaque existing inside a blood vessel (hereinafter also simply called the “second laser light”).

The wavelength of the first laser light emitted from the first laser light source 101 is not particularly limited insofar as the wavelength is selectively absorbed by plaque existing inside a blood vessel, and it is possible to use any wavelength having such characteristics. For example, according to research using rabbits with hereditary hypercholesterolemia (WHHLMI rabbits), it is known that plaque selectively absorbs light with a wavelength from 5.64 μm to 5.84 μm (for example, see K. Hashimura, K. Ishii and K. Awazu, “Selective removal of atherosclerotic plaque with a quantum cascade laser in the 5.7 μm wavelength range”, Japanese Journal of Applied Physics, 54(2015), p. 112701). Accordingly, the wavelength of the first laser light emitted from the first laser light source 101 preferably is inside the range from 5.63 μm to 5.84 μm. More preferably, the wavelength of the first laser light is approximately 5.75 μm.

Similarly, the wavelength of the second laser light emitted from the second laser light source 103 is not particularly limited insofar as the wavelength is selectively absorbed by calcified plaque existing inside a blood vessel, and it is possible to use any wavelength having such characteristics. In a calcified lesion inside a blood vessel, it is thought that calcium phosphate is being deposited inside the blood vessel. Herein, from knowledge related to the group frequencies in a vibrational spectrum of molecules (for example, see G. Socrates, “Infrared and Raman characteristic group frequencies”) and the infrared absorption spectrum of a sample including calcium phosphate (for example, see JP 2007-31226A), it is known that calcified plaque and calcium phosphate selectively absorb light with a wavelength from 3.76 μm to 3.96 μm or light with a wavelength from 7.55 μm to 9.26 μm. Accordingly, the wavelength of the second laser light emitted from the second laser light source 103 preferably is inside the wavelength range from 3.76 μm to 3.96 μm or the wavelength range from 7.55 μm to 9.26 μm. More preferably, the wavelength of the second laser light is approximately 3.86 μm.

The type of the first laser light source 101 and the second laser light source 103 that emit laser light of wavelengths like the above is not particularly limited, but it is preferable to use a laser light source using a semiconductor with a small emission area. In the past, since the excimer laser that has been used in ECLA has a relatively large laser emission area (such as 9 mm×25 mm, for example), it has not been possible to condense the laser light into a small spot even if a condenser lens is used, it has not been possible to connect the laser to a single-mode optical fiber, and it has been necessary to connect the laser to an image guide fiber. For this reason, narrowing the diameter of the catheter itself has not been easy, and cases in which application is difficult exist. On the other hand, for the wavelength band of the first laser light and the second laser light that are the subject of the present embodiment, it is possible to use a laser light source using a semiconductor. For a laser light source using a semiconductor, the laser emission area is microscopic compared to an excimer laser, and it is also possible to connect to a single-mode optical fiber. With this arrangement, it becomes possible to attain a narrower diameter of the optical fiber to use, making it possible to increase the number of cases where the medical laser irradiation device 10 according to the present embodiment is applicable.

As the laser light source using a semiconductor like the above, it is possible to use a publicly available laser insofar as the laser is capable of emitting laser light of a wavelength band like the above. For example, it is preferable to use a quantum cascade laser light source, or a light source combining a solid-state microlaser including a solid-state gain medium with a wavelength conversion element.

Due to recent technological progress, a quantum cascade laser light source is capable of emitting laser light on a wavelength from approximately 3 μm to approximately 11 μm, and by using such a light source, it becomes possible to easily use each laser light corresponding to the two varieties of plaque states like the above.

Also, as disclosed in JP H7-112082B and the like, for example, a solid-state microlaser including a solid-state gain medium is able to emit laser light of various wavelengths by changing the type of solid-state gain medium used. For example, by using the semiconductor neodymium-doped yttrium aluminum garnet (Nd:YAG) crystal as the solid-state gain medium, ultrashort pulse high-output pulsed-laser light with a wavelength of 1064 nm can be obtained. By combining such pulsed-laser light with a publicly available wavelength conversion element such as MgO-doped polarization-inverted lithium niobate crystal (PPMgLN), for example, it becomes possible to easily use each laser light corresponding to the two varieties of plaque states like the above.

Also, for a quantum cascade laser or a light source combining a solid-state microlaser with a wavelength conversion element (particularly, a light source combining a solid-state microlaser with a wavelength conversion element) like the above, it is extremely easy to attain a miniaturization the laser light source itself, which also makes it possible to attain a miniaturization of the medical laser irradiation device 10 according to the present embodiment itself.

The first laser light and the second laser light emitted from various types of laser light sources like the above are guided by publicly available optical elements such as a mirror M and a beam-combining mirror Com, and are connected to the optical fiber 105.

The optical fiber 105 according to the present embodiment coaxially guides the first laser light and the second laser light like the above, and at least a part of which is inserted into a blood vessel. In the medical laser irradiation device 10 according to the present embodiment, since laser light in the near-infrared to mid-infrared wavelength band like the above is used, such laser light can be guided with a single-mode optical fiber, making it possible to attain a narrower diameter of the optical fiber itself. Accordingly, in the medical laser irradiation device 10 according to the present embodiment, it is desirable for such an optical fiber 105 to have an outer diameter of 0.9 mm or less, including various auxiliary structures such as a sheath. By making the outer diameter of the optical fiber 105 be 0.9 mm or less, it becomes possible to further increase the number of cases where the medical laser irradiation device 10 according to the present embodiment is applicable.

Also, for the optical fiber 105 according to the present embodiment, any optical fiber may be used insofar as it is possible to guide light in the near-infrared to mid-infrared wavelength band like the above, but it is preferable to use a chalcogenide optical fiber as such optical fiber. A chalcogenide optical fiber is an optical fiber having a compound (chalcogenide glass) containing large amounts of chalcogen elements in the narrow sense such as sulfur (S), selenium (Se), and tellurium (Te) as the core, and is capable of propagating light in the wavelength band from 1.1 μm to 6.5 μm, including the wavelength band focused on by the present embodiment, through the same optical fiber. Consequently, by using a chalcogenide optical fiber as the optical fiber 105, it becomes possible to coaxially propagate the first laser light and the second laser light like the above easily.

The computational processing unit 107 is realized by a central processing unit (CPU), read-only memory (ROM), random access memory (RAM), and the like, for example. The computational processing unit 107 controls the driving of the first laser light source 101 and the second laser light source 103 according to user operations and the like performed by the user of the medical laser irradiation device 10. With this arrangement, more fine-grained control of the driving of the laser light sources, such as preventing unnecessary laser irradiation and the like, may be achieved. Also, the computational processing unit 107 according to the present embodiment is capable of performing various analysis processes on the basis of various information obtained from various configurations of the medical laser irradiation device 10 including the first laser light source 101 and the second laser light source 103. In addition, the computational processing unit 107 is also able to control the display of the display unit 109 that may be provided in the medical laser irradiation device 10. A detailed configuration of such a computational processing unit 107 will be described further below.

The display unit 109 is a unit including any of various types of displays provided in the medical laser irradiation device 10 according to the present embodiment. On such a display unit 109, various information related to the driving conditions of the medical laser irradiation device 10, such as the laser output of each laser light source and the like, is displayed. By referring to the various information output to such a display unit 109, the user of the medical laser irradiation device 10 becomes able to easily grasp the driven state of the medical laser irradiation device 10 and the like on the spot.

Note that in the medical laser irradiation device 10 according to the present embodiment, to remove plaque existing inside a blood vessel more efficiently, laser light in the near-infrared to mid-infrared wavelength band is used, and a specific optical fiber for propagating laser light in such a wavelength band is used.

Note that since the medical laser irradiation device 10 according to the present embodiment whose overall configuration is illustrated in FIG. 1 is a device using light in the near-infrared to mid-infrared wavelength band as above, it is preferable to use optical elements applicable to light in such a wavelength band. Such optical elements are not particularly limited, but may be exemplified by CaF optical elements, polyethylene optical elements, and the like, for example. By using at least one of CaF optical elements or polyethylene optical elements to configure the optical system of the medical laser irradiation device 10 according to the present embodiment, it becomes possible to guide light in the wavelength band like the above more reliably.

The above describes the overall configuration of the medical laser irradiation device 10 according to the present embodiment while referring to FIG. 1.

### [Irradiation Unit]

In the medical laser irradiation device 10 according to the present embodiment, to make the irradiation direction and the condensed state of laser light radiating from the front end of the optical fiber 105 variable, as schematically illustrated in FIG. 2, on the front end of the optical fiber 105, it is preferable to provide an irradiation unit 121 for irradiating the interior of a blood vessel with light guided by the optical fiber.

By providing such an irradiation unit 121, it becomes possible to set the irradiation direction and the condensed state of the laser light radiating from the front end of the optical fiber 105 to a desired state, making it possible to radiate the laser light more effectively in conjunction with the state of attached plaque inside a blood vessel.

As illustrated in FIG. 3A for example, an irradiation unit 121 provided with a lens (for example, a CaF lens) L for condensing light guided by the optical fiber 105 may be prepared and mounted onto the front end of the optical fiber 105. With this arrangement, it becomes possible to condense and also radiate the light guided by the optical fiber 105 toward plaque from the forward optical axis direction of the optical fiber 105. As a result, it becomes possible to irradiate plaque positioned in front of the irradiation unit 121 with laser light effectively. For this reason, it becomes possible to irradiate even plaque inside a blood vessel in a completely blocked state with laser light easily.

Additionally, as illustrated in FIG. 3B for example, by preparing the irradiation unit 121 using a special lens such as a Fresnel lens or a ball lens or the like (for example, a polyethylene Fresnel lens or a CaF ball lens), it is also possible to radiate light guided by the optical fiber 105 from the side of the optical fiber 105. By using the irradiation unit 121 as illustrated in FIG. 3B, it is possible to radiate laser light from the side face of the optical fiber 105, thereby making it possible to irradiate plaque with laser light and disintegrate the plaque even in cases in which the interior of the blood vessel is extremely constricted.

Note that it is preferable for the irradiation units 121 as illustrated in FIGS. 3A and 3B to be detachably provided on the front end of the optical fiber 105.

### [Guide Wire Insertion Hole]

In the case of using the medical laser irradiation device 10 according to the present embodiment to remove plaque existing inside a blood vessel, disposing the optical fiber 105 with respect to the lesion appropriately is important. For this reason, in the medical laser irradiation device 10 according to the present embodiment, a guide wire insertion hole into which a guide wire that guides the optical fiber 105 to a desired position inside the blood vessel is inserted is preferably provided at an appropriate position.

As illustrated schematically in FIG. 4A for example, a guide wire insertion hole 131 into which a guide wire is inserted is provided in parallel with the optical fiber 105, and it is possible to sheathe the guide wire insertion hole 131 and the optical fiber 105 in a single bundle. Even in such a case, it is preferable for the maximum combined outer diameter of the guide wire insertion hole 131 and the optical fiber 105 to be 0.9 mm or less. By providing the guide wire insertion hole 131 as illustrated in FIG. 4A, it becomes possible to realize what is called an over-the-wire (OTW) medical laser irradiation device 10.

Also, as illustrated schematically in FIG. 4B for example, it is also possible to provide the guide wire insertion hole 131 on at least one of the outer side of a sheathe covering the optical fiber 105 or the outer side of the front end of the optical fiber 105. Even in such a case, it is preferable for the maximum combined outer diameter of the guide wire insertion hole 131 and the optical fiber 105 to be 0.9 mm or less. By providing the guide wire insertion hole 131 as illustrated in FIG. 4B, it becomes possible to achieve what is called a rapid exchange (RX) medical laser irradiation device 10.

### [Configuration of Computational Processing Unit 107]

Next, one example of the configuration of the computational processing unit 107 according to the present embodiment will be described while referring to FIG. 5.

As illustrated in FIG. 5, it is preferable for the computational processing unit 107 according to the present embodiment to be mainly provided with a control section 151, an analysis section 153, a display control section 155, and a storage section 157.

The control section 151 is realized by a CPU, ROM, RAM, a communication device, and the like, for example. The control section 151, by controlling the driving of each of the first laser light source 101 and the second laser light source 103 in the medical laser irradiation device 10 according to the present embodiment, achieves emission control of the first laser light and emission control of the second laser light. With this arrangement, the medical laser irradiation device 10 according to the present embodiment becomes able to emit each of the first laser light and the second laser light at a desired laser output and at a desired timing. As a result, in the medical laser irradiation device 10 according to the present embodiment, it becomes possible to emit only the first laser light or the second laser light alone, or emit each of the first laser light and the second laser light simultaneously at a predetermined laser output. Furthermore, in the medical laser irradiation device 10 according to the present embodiment, by controlling the emission timings of each laser light, it becomes possible to prevent laser light irradiation at unwanted timings and achieve safer laser irradiation.

When performing various types of control like the above, the control section 151 is able to acquire various information from each piece of equipment and each unit included in the optical system of the medical laser irradiation device 10. For example, the control section 151 is able to acquire information related to the output of laser light emitted from the first laser light source 101 and the second laser light source 103 and acquire information related to the reflected light intensity and the like of each laser light in the optical system, and use the acquired information for various types of control like the above.

For example, the control section 151 is able to output acquired information related to the reflected light intensity and the like of each laser light in the optical system, information related to an optical tomographic image, and the like to the analysis section 153 described later to analyze the degree of calcification of plaque existing inside a blood vessel. In this case, when a result of analyzing the degree of calcification of plaque is acquired from the analysis section 153, the control section 151 controls the output of each of the first laser light and the second laser light according to the obtained analysis result. With this arrangement, it becomes possible to control the intensity of each laser light to suit the state (degree of calcification) of plaque existing inside a blood vessel, and control the irradiation time according to the thickness of the plaque. At this point, the control section 151 is able to execute various types of control processes, such as deciding the intensity of each laser light according to the degree of calcification and the like, while referencing various databases or the like stored in the storage section 157 described later or the like.

Also, through the display control section 155, the control section 151 is able to cause the display unit 109 to display various information acquired from each piece of equipment and each unit included in the optical system of the medical laser irradiation device 10, information expressing the result of controlling each laser light source, and the like.

The analysis section 153 is realized by a CPU, ROM, RAM, and the like, for example. The analysis section 153 is a processing section that analyzes the state of plaque (more specifically, the degree of calcification of plaque) existing inside a blood vessel.

As mentioned earlier, plaque existing inside a blood vessel selectively absorbs light having a certain specific wavelength, and calcified plaque also selectively absorbs light having a certain specific wavelength. Consequently, by varying the allocation of irradiating laser light according to the degree of calcification of plaque, it becomes possible to cause plaque and calcified plaque to absorb laser light appropriately, and remove these plaques and the like efficiently.

Accordingly, in the analysis section 153 according to the present embodiment, the degree of calcification of plaque is analyzed on the basis of the absorptance or reflectance of the first laser light and the absorptance or reflectance of the second laser light acquired from the control section 151, for example. If the degree of calcification of plaque existing inside a blood vessel is low, the absorptance of the first laser light becomes large, and the higher the degree of calcification of plaque existing inside a blood vessel, the greater the absorptance of the second laser light. Accordingly, by focusing on the absorptance or reflectance of each laser light, it becomes possible to analyze the degree of calcification of plaque existing inside a blood vessel.

The specific method of quantifying the degree of calcification of plaque is not particularly limited, and it is sufficient to quantify appropriately according to a publicly available method using the absorptance (or reflectance) of each laser light.

The display control section 155 is realized by a CPU, ROM, RAM, an output device, a communication device, and the like, for example. The display control section 155 controls the display when displaying the results of various types of control output from the control section 151 and various information related to the various analysis results and the like output from the analysis section 153 on an output device such as a display provided in the display unit 109 of the medical laser irradiation device 10, an output device provided externally to the medical laser irradiation device 10, or the like. With this arrangement, the operator of the medical laser irradiation device 10 becomes able to understand various results on the spot.

The storage section 157 is one example of a storage device provided in the computational processing unit 107 of the medical laser irradiation device 10, and is realized by RAM, a storage device, or the like provided in the computational processing unit 107. In the storage section 157, various databases that the computational processing unit 107 according to the present embodiment uses when performing various processes are recorded. In addition, various history information may also be recorded in the storage section 157. Furthermore, in the storage section 157, various parameters, partial results of processes, and the like that need to be saved when the computational processing unit 107 according to the present embodiment executes some kind of process, or various databases and programs or the like, are recorded as appropriate. The control section 151, the analysis section 153, the display control section 155, and the like are able to perform read/write processes freely with respect to the storage section 157.

In the above, one example of the function of the computational processing unit 107 according to the present embodiment has been shown. The respective structural elements described in the above may be configured by using general-purpose components and circuits, or may be configured by hardware specialized for the functions of the respective structural elements. Moreover, a CPU or the like may perform all the functions of the respective structural elements. Therefore, in accordance with the technical level at the time of carrying out the present embodiment, it is possible to appropriately change the configuration to be used.

In this connection, it is possible to produce computer programs for realizing each function of the computational processing unit according to the present embodiment as described in the above, and to mount them in a personal computer and the like. Moreover, it is possible to provide also a computer-readable recording medium in which such computer programs are stored. The recording media may be, for example, a magnetic disc, an optical disc, a magneto-optical disc, flash memory, or the like. Moreover, the above-described computer programs may be distributed, for example, through networks, without using a recording medium.

### [Optical Block Diagram]

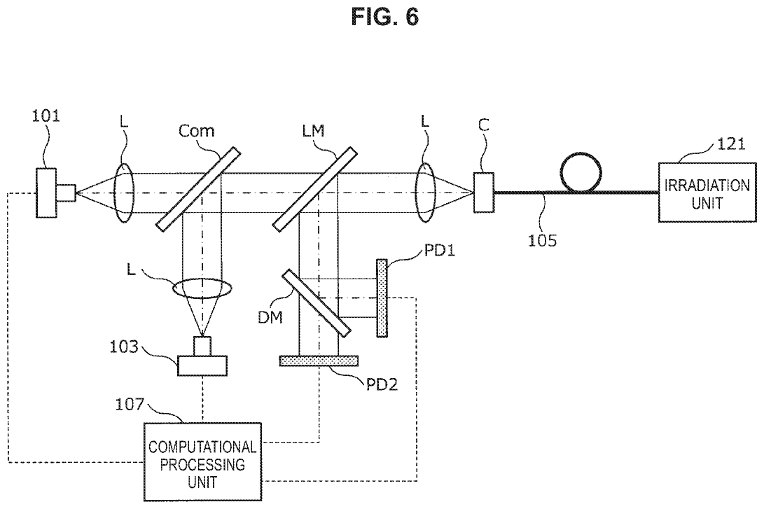

Next, one example of an optical block diagram of the medical laser irradiation device 10 according to the present embodiment will be described while referring to FIG. 6. FIG. 6 is a block diagram schematically illustrating one example of the optical configuration of the medical laser irradiation device according to the present embodiment.

FIG. 6 is one example of an optical block diagram of the medical laser irradiation device 10 having the configuration illustrated in FIG. 1.

Under control by the computational processing unit 107, the first laser light emitted from the first laser light source 101 and the second laser light emitted from the second laser light source 103 are collimated by the lens L, and then combined with each other by the beam-combining mirror Com. The combined first laser light and second laser light are transmitted through a low-reflection mirror LM, and then is condensed into a connector C by the lens L, and connected to the optical fiber 105. The first laser light and the second laser light irradiate plaque through the irradiation unit 121, and plaque is removed by these laser lights.

Also, part of the first laser light and second laser light is reflected by plaque, and the reflected light of each laser light reaches the low-reflection mirror LM through the optical fiber 105, and is split by the low-reflection mirror LM onto an optical path different from the optical path going toward each laser light source. The split reflected light is split by a dichroic mirror DM into reflected light of the first laser light and reflected light of the second laser light. The reflected light of the first laser light is detected by a photodetector PD1 that detects the reflected light of the first laser light, and the reflected light of the second laser light is detected by a photodetector PD2 that detects the reflected light of the second laser light. The result of detecting each reflected light by each of the photodetectors PD1 and PD2 is output to the computational processing unit 107 and used in various processes.

The above describes one example of an optical block diagram of the medical laser irradiation device 10 according to the present embodiment while referring to FIG. 6.

### <Hardware Configuration>

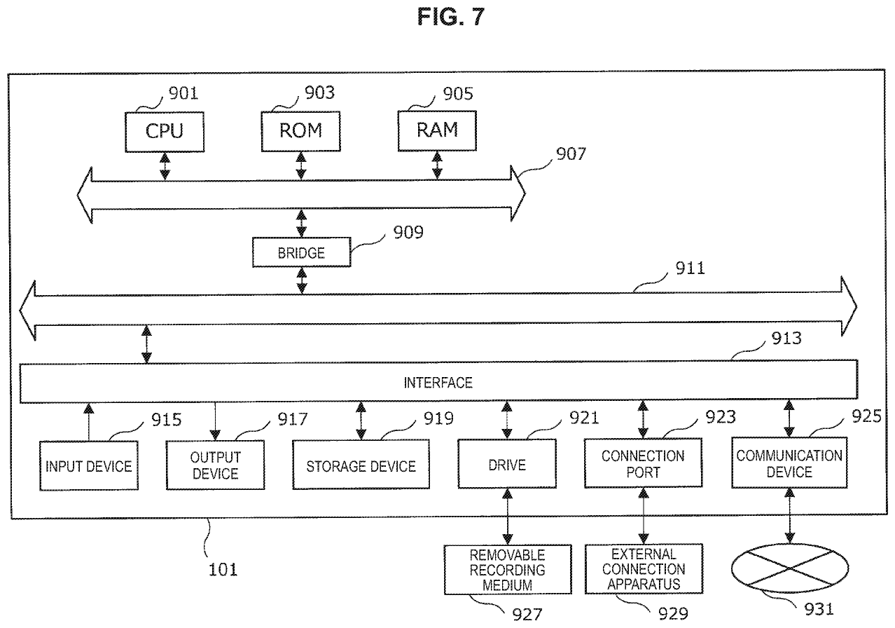

Next, the hardware configuration of the computational processing unit 107 according to the embodiment of the present disclosure will be described in detail with reference to FIG. 7. FIG. 7 is a block diagram for describing the hardware configuration of the computational processing unit 107 according to the embodiment of the present disclosure.

Hereinafter, the computational processing unit 107 mainly includes a CPU 901, a ROM 903, and a RAM 905. Furthermore, the computational processing unit 107 also includes a host bus 907, a bridge 909, an external bus 911, an interface 913, an input device 915, an output device 917, a storage device 919, a drive 921, a connection port 923, and a communication device 925.

The CPU 901 serves as a main computational processing apparatus and a control apparatus, and controls the overall operation or a part of the operation of the computational processing unit 107 according to various programs recorded in the ROM 903, the RAM 905, the storage device 919, or a removable recording medium 927. The ROM 903 stores programs, operation parameters, and the like used by the CPU 901. The RAM 905 primarily stores programs used the CPU 901 and parameters and the like varying as appropriate during the execution of the programs. These are connected with each other via the host bus 907 including an internal bus such as a CPU bus.

The host bus 907 is connected to the external bus 911 such as a PCI (Peripheral Component Interconnect/Interface) bus via the bridge 909.

The input device 915 is an operation means operated by a user, such as a mouse, a keyboard, a touch panel, buttons, a switch and a lever, for example. Also, the input device 915 may be a remote control means (a so-called remote controller) using, for example, infrared light or other radio waves, or may be an external connection apparatus 929 such as a mobile phone or a PDA conforming to the operation of the computational processing unit 107. Furthermore, the input device 915 generates an input signal on the basis of, for example, information which is input by a user with the above operation means, and includes an input control circuit or the like for outputting the input signal to the CPU 901. The user can input various data to the computational processing unit 107 and can instruct the computational processing unit 107 to perform various types of processing by operating this input device 915.

The output device 917 includes a device capable of visually or audibly notifying a user of acquired information. Such a device includes a display device such as a CRT display device, a liquid crystal display device, a plasma display device, an EL display device and a lamp, an audio output device such as a speaker and a headphone, a printer, a mobile phone, a facsimile machine, and the like. For example, the output device 917 outputs a result obtained by various types of processing performed by the computational processing unit 107. Specifically, the display device displays, in the form of text or images, a result obtained by various types of processing performed by the computational processing unit 107. On the other hand, the audio output device converts an audio signal including reproduced audio data, sound data, and the like into an analog signal, and outputs the analog signal.

The storage device 919 is a device for storing data configured as an example of a storage unit of the computational processing unit 107. The storage device 919 includes, for example, a magnetic storage device such as an HDD (Hard Disk Drive), a semiconductor storage device, an optical storage device, a magneto-optical storage device, or the like. This storage device 919 stores programs to be executed by the CPU 901 and various types of data, externally obtained various types of data, and the like.

The drive 921 is a reader/writer for a recording medium, and is built in the computational processing unit 107 or attached externally thereto. The drive 921 reads information recorded in the attached removable recording medium 927 such as a magnetic disk, an optical disc, a magneto-optical disk, or a semiconductor memory, and outputs the read information to the RAM 905. Furthermore, the drive 921 can write records in the attached removable recording medium 927 such as a magnetic disk, an optical disc, a magneto-optical disk, or a semiconductor memory. The removable recording medium 927 is, for example, a DVD medium, an HD-DVD medium, a Blu-ray (registered trademark) medium, or the like. In addition, the removable recording medium 927 may be a CompactFlash (CF; registered trademark), a flash memory, an SD memory card (Secure Digital Memory Card), or the like. Further, the removable recording medium 927 may be, for example, an IC card (Integrated Circuit Card) equipped with a non-contact IC chip, an electronic appliance, or the like.

The connection port 923 is a port for allowing devices to directly connect to the computational processing unit 107. Examples of the connection port 923 include a USB (Universal Serial Bus) port, an IEEE1394 port, a SCSI (Small Computer System Interface) port, and the like. Other examples of the connection port 923 include an RS-232C port, an optical audio terminal, a High-Definition Multimedia Interface (HDMI, registered trademark) port, and the like. By connecting the external connection apparatus 929 to this connection port 923, the computational processing unit 107 directly acquires various types of data from the external connection apparatus 929 and provides various types of data to the external connection apparatus 929.

The communication device 925 is a communication interface including, for example, a communication device or the like for connecting to a communication network 931. The communication device 925 is, for example, a communication card or the like for a wired or wireless LAN (Local Area Network), Bluetooth (registered trademark), or WUSB (Wireless USB). Further, the communication device 925 may be a router for optical communication, a router for ADSL (Asymmetric Digital Subscriber Line), a modem for various types of communication, or the like. This communication device 925 can transmit and receive signals and the like in accordance with a predetermined protocol, for example, such as TCP/IP on the Internet and with other communication devices, for example. In addition, the communication network 931 connected to the communication device 925 includes a network and the like which is connected in a wire or wireless manner, and may be, for example, the Internet, a home LAN, infrared communication, radio wave communication, satellite communication, or the like.

The above illustrates an example of the hardware configuration capable of realizing the functions of the computational processing unit 107 according to the embodiment of the present disclosure. Each of the above structural elements may be realized using a general-purpose member, or may also be realized using hardware specialized in the function of each structural element. Consequently, it is possible to appropriately modify the hardware configuration to be used according to the technical level at the time of carrying out the present embodiment.

### CONCLUSION

As described above, in the medical laser irradiation device 10 according to an embodiment of the present disclosure, by using first laser light that is selectively absorbed by plaque, it becomes possible to perform laser irradiation highly efficiently and safely. Also, in the medical laser irradiation device 10 according to an embodiment of the present disclosure, by jointly using second laser light that is selectively above by calcified plaque, it becomes possible to treat calcified lesions. Also, by adjusting the combination ratio of the first laser light and the second laser light according to the state of plaque, it becomes possible to achieve effective treatment. The adjustment of the combination ratio of each of the laser lights may be performed in advance prior to treatment, but may also be performed dynamically while monitoring the status of the treatment.

Also, in the medical laser irradiation device 10 according to an embodiment of the present disclosure, since it is possible to use a light source having a small luminous point, by using a condenser lens, it becomes possible to cause laser light to be incident with high efficiency, even with respect to an optical fiber of narrow diameter (single-mode optical fiber). As a result, it becomes possible to attain a narrower diameter of the optical fiber to use, making it also possible to treat a chronic total occlusion.

Also, since it is possible to use a light source using a semiconductor as the first laser light source 101 and the second laser light source, it also becomes possible to make effective use of the operating room by saving space and to reduce the burden on the patient and physician by a noise-reducing effect.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

A medical laser irradiation device including:

a first laser light source configured to emit a first laser light having a wavelength band that is selectively absorbed by plaque existing inside a blood vessel of a living body;

a second laser light source configured to emit a second laser light having a wavelength band that is selectively absorbed by the plaque calcified and existing inside the blood vessel; and

an optical fiber configured to coaxially guide the first laser light and the second laser light, and at least a part of which is inserted into the blood vessel.

(2)

The medical laser irradiation device according to (1), further including:

a control section configured to control an emission of the first laser light from the first laser light source and to control an emission of the second laser light from the second laser light source; and

an analysis section configured to analyze a degree of calcification of the plaque, wherein

the control section controls an output of each of the first laser light and the second laser light according to a result of analyzing the degree of calcification of the plaque by the analysis section.

(3)

The medical laser irradiation device according to (2), in which

the analysis section analyzes the degree of calcification of the plaque on the basis of at least an absorptance or a reflectance of the first laser light and an absorptance or a reflectance of the second laser light.

(4)

The medical laser irradiation device according to any one of (1) to (3), in which

on a front end of the optical fiber, an irradiation unit for irradiating the inside of the blood vessel with light guided by the optical fiber is provided.

(5)

The medical laser irradiation device according to (4), in which

the irradiation unit radiates light guided by the optical fiber from a forward optical axis direction of the optical fiber.

(6)

The medical laser irradiation device according to (4), in which

the irradiation unit radiates light guided by the optical fiber from a side of the optical fiber.

(7)

The medical laser irradiation device according to any one of (4) to (6), in which

the irradiation unit is detachably provided on the front end of the optical fiber.

(8)

The medical laser irradiation device according to any one of (1) to (7), in which

a wavelength of the first laser light is inside a range from 5.63 μm to 5.84 μm, and

a wavelength of the second laser light is inside a range from 3.76 μm to 3.96 μm or inside a range from 7.55 μm to 9.26 μm.

(9)

The medical laser irradiation device according to any one of (1) to (8), in which

each of the first laser light source and the second laser light source is a laser light source using a semiconductor.

(10)

The medical laser irradiation device according to any one of (1) to (8), in which

each of the first laser light source and the second laser light source is a quantum cascade light source or a light source combining a solid-state microlaser including a solid-state gain medium with a wavelength conversion element.

(11)

The medical laser irradiation device according to any one of (1) to (10), in which

the optical fiber is a chalcogenide optical fiber.

(12)

The medical laser irradiation device according to any one of (1) to (11), in which

an outer diameter of the optical fiber is 0.9 mm or less.

(13)

The medical laser irradiation device according to any one of (1) to (12), in which

an optical system is configured using at least any one of a CaF optical element or a polyethylene optical element.

(14)

The medical laser irradiation device according to any one of (1) to (13), further comprising:

a guide wire insertion hole into which a guide wire configured to guide the optical fiber to a desired position inside the blood vessel is inserted.

(15)

The medical laser irradiation device according to (14), in which

the guide wire insertion hole is provided in parallel with the optical fiber, and is sheathed together with the optical fiber.

(16)

The medical laser irradiation device according to (14), in which

the guide wire insertion hole is provided on at least any of a sheathe covering the optical fiber or an outer side of a front end of the optical fiber.

(17)

A medical laser irradiation method including:

guiding a first laser light emitted from a first laser light source configured to emit the first laser light having a wavelength band that is selectively absorbed by plaque existing inside a blood vessel of a living body and a second laser light emitted from a second laser light source configured to emit the second laser light having a wavelength band that is selectively absorbed by the plaque calcified and existing inside the blood vessel with an optical fiber configured to coaxially guide the first laser light and the second laser light; and

irradiating the inside of the blood vessel with at least any one of the first laser light or the second laser light from a front end of the optical fiber, at least a part of which is inserted into the blood vessel.

### REFERENCE SIGNS LIST

10 medical laser irradiation device
101 first laser light source
103 second laser light source
105 optical fiber
107 computational processing unit
109 display unit
121, 123 irradiation unit
131 guide wire insertion hole
151 control section
153 analysis section
155 display control section
157 storage section

1. A medical laser irradiation device comprising:
a first laser light source configured to emit a first laser light having a wavelength band that is selectively absorbed by plaque existing inside a blood vessel of a living body;
a second laser light source configured to emit a second laser light having a wavelength band that is selectively absorbed by the plaque calcified and existing inside the blood vessel; and
an optical fiber configured to coaxially guide the first laser light and the second laser light, and at least a part of which is inserted into the blood vessel.

2. The medical laser irradiation device according to claim 1, further comprising:
a control section configured to control an emission of the first laser light from the first laser light source and to control an emission of the second laser light from the second laser light source; and
an analysis section configured to analyze a degree of calcification of the plaque, wherein
the control section controls an output of each of the first laser light and the second laser light according to a result of analyzing the degree of calcification of the plaque by the analysis section.

3. The medical laser irradiation device according to claim 2, wherein
the analysis section analyzes the degree of calcification of the plaque on a basis of at least an absorptance or a reflectance of the first laser light and an absorptance or a reflectance of the second laser light.

4. The medical laser irradiation device according to claim 1, wherein
on a front end of the optical fiber, an irradiation unit for irradiating the inside of the blood vessel with light guided by the optical fiber is provided.

5. The medical laser irradiation device according to claim 4, wherein
the irradiation unit radiates light guided by the optical fiber from a forward optical axis direction of the optical fiber.

6. The medical laser irradiation device according to claim 4, wherein
the irradiation unit radiates light guided by the optical fiber from a side of the optical fiber.

7. The medical laser irradiation device according to claim 4, wherein
the irradiation unit is detachably provided on the front end of the optical fiber.

8. The medical laser irradiation device according to claim 1, wherein
a wavelength of the first laser light is inside a range from 5.63 μm to 5.84 μm, and
a wavelength of the second laser light is inside a range from 3.76 μm to 3.96 μm or inside a range from 7.55 μm to 9.26 μm.

9. The medical laser irradiation device according to claim 1, wherein
each of the first laser light source and the second laser light source is a laser light source using a semiconductor.

10. The medical laser irradiation device according to claim 1, wherein
each of the first laser light source and the second laser light source is a quantum cascade light source or a light source combining a solid-state microlaser including a solid-state gain medium with a wavelength conversion element.

11. The medical laser irradiation device according to claim 1, wherein
the optical fiber is a chalcogenide optical fiber.

12. The medical laser irradiation device according to claim 1, wherein
an outer diameter of the optical fiber is 0.9 mm or less.

13. The medical laser irradiation device according to claim 1, wherein
an optical system is configured using at least any one of a CaF optical element or a polyethylene optical element.

14. The medical laser irradiation device according to claim 1, further comprising:
a guide wire insertion hole into which a guide wire configured to guide the optical fiber to a desired position inside the blood vessel is inserted.

15. The medical laser irradiation device according to claim 14, wherein
the guide wire insertion hole is provided in parallel with the optical fiber, and is sheathed together with the optical fiber.

16. The medical laser irradiation device according to claim 14, wherein
the guide wire insertion hole is provided on at least any of a sheathe covering the optical fiber or an outer side of a front end of the optical fiber.

17. A medical laser irradiation method comprising:
guiding a first laser light emitted from a first laser light source configured to emit the first laser light having a wavelength band that is selectively absorbed by plaque existing inside a blood vessel of a living body and a second laser light emitted from a second laser light source configured to emit the second laser light having a wavelength band that is selectively absorbed by the plaque calcified and existing inside the blood vessel with an optical fiber configured to coaxially guide the first laser light and the second laser light; and
irradiating the inside of the blood vessel with at least any one of the first laser light or the second laser light from a front end of the optical fiber, at least a part of which is inserted into the blood vessel.
